(19) United States
(12) Patent Application Publication
(10) Pub. No.: US 2016/0022172 A1
(43) Pub. Date: Jan. 28, 2016

(54) SYSTEMS AND METHODS FOR FLUID TESTING

(71) Applicant: Lifeloc Technologies, Inc., Wheat Ridge, CO (US)

(72) Inventor: Jorgen Frandsen, Broomfield, CO (US)

(21) Appl. No.: 14/339,345

(22) Filed: Jul. 23, 2014

(51) Int. Cl. A61B 5/08; A61B 5/097; G01N 27/30

(52) U.S. Cl. CPC A61B 5/082; G01N 27/30; A61B 5/097

(57) ABSTRACT

Implementations disclosed herein provide a method of determining a quantity of an electrochemically convertible substance in a fluid sample, the method comprising introducing the fluid sample into an electrochemical sensor, wherein at least a portion of the fluid sample is electrochemically converted to produce an electrical output from the electrochemical sensor, measuring the electrical output from the electrochemical sensor on a periodic basis to produce sensor measurements, inputting a first subset of the sensor measurements into a first computation to yield first computation analysis results, inputting a second subset of the sensor measurements and the first computation analysis results into a second computation to yield second computation analysis results, and calculating the quantity of the electrochemically convertible substance in the fluid sample by applying a third computation to the first computation analysis results and the second computation analysis results.

### BACKGROUND

Handheld breath alcohol testing devices are useful in roadside estimation of blood alcohol level of drivers. Electrochemical sensors are commonly used in these devices to detect a concentration of alcohol in a sample of fluid. The sample of fluid (e.g., breath samples, gases, liquids, and mixtures thereof) is introduced into the electrochemical sensor and a current is generated by the oxidation of the alcohol within the fluid. The electrical output from the electrochemical sensor increases from an initial value to a peak value and then decreases back to or near the initial value. These output amplitude measurements, plotted over time, form an output curve, which may be used to estimate the concentration of alcohol in the fluid sample.

### SUMMARY

Implementations described and claimed herein provide a method of determining a quantity of an electrochemically convertible substance in a fluid sample, the method comprising introducing the fluid sample into an electrochemical sensor, wherein at least a portion of the fluid sample is electrochemically converted to produce an electrical output from the electrochemical sensor, measuring the electrical output from the electrochemical sensor on a periodic basis to produce sensor measurements, inputting a first subset of the sensor measurements into a first computation to yield first computation analysis results, inputting a second subset of the sensor measurements and the first computation analysis results into a second computation to yield second computation analysis results, and calculating the quantity of the electrochemically convertible substance in the fluid sample by applying a third computation to the first computation analysis results and the second computation analysis results.

This Summary is provided to introduce an election of concepts in a simplified form that are further described below in the Detailed Description. This Summary is not intended to identify key features or essential features of the claimed subject matter, nor is it intended to limit the scope of the claimed subject matter. Other features, details, utilities, and advantages of the claimed subject matter will be apparent from the following more particular written Detailed Description of various implementations as further illustrated in the accompanying drawings and defined in the appended claims.

### BRIEF DESCRIPTION OF THE DRAWINGS

FIG. 1 is a front perspective view of an example breath alcohol content device incorporating the fluid testing computations disclosed herein.

FIG. 2 is a block diagram showing an example electronic circuit of a fluid analysis apparatus.

FIG. 3 is a graph of example data values for fuel cell output of an example breath alcohol content device.

FIG. 4 is a second graph of example data values for fuel cell output of an example breath alcohol content device.

FIG. 5 is a third natural logarithmic graph of example data values for fuel cell output of an example breath alcohol content device.

FIG. 6 is a flowchart of example operations for determining the quantity of an electrochemically convertible substance in a fluid sample.

### DETAILED DESCRIPTION

The technology disclosed herein includes a method for determining the quantity of an electrochemically convertible substance in a fluid sample with a breath alcohol content device (or other fluid analysis apparatus). The fluid may be a gas, liquid, or a gas/liquid mixture (e.g., a breath sample).

The disclosed technology involves measuring maximum fuel cell sensor output to quantify alcohol content of the fluid sample. Specifically, the total charge delivered in the fuel cell sensor is measured by integrating the current over the full time duration of the current flow from the sensor. However, for practical reasons, the full time duration is not used. The integration of current over a defined period of time results in an estimated area of a current versus time curve, whereas determining the total actual area would take an infinite time since the current drops off approximately exponentially and never actually reaches zero. Therefore, because it is impractical to wait an infinite time, the disclosed technology terminates measurement while current is still flowing. As a result, the measurement is based on finite data collected up to a certain point in time. The truncated data set is analyzed to determine the total area of the entire data set as if it had been collected over an infinite period of time.

To perform total area estimation, curve fitting a function to all of the collected data and then performing an analytical integration of the function to find the area from time zero to infinity may be used. However, this analytical fitting approach may not fit the data to the function accurately and an inaccurate fit results in errors. There may also be a tradeoff in function fitting the early parts of the sensor output curve at the same time as accurately fitting the later part of the sensor output curve. The function fitting error of the later part of the curve due to fitting errors of the first part affects the accuracy of the estimate of the area of the extrapolated tail of the curve. Higher extrapolated tail area calculation accuracy may be achieved by performing a function fit using data from the later parts of the collected data curve. The presently disclosed technology analyzes the collected data over a finite period of time and finds the total area of the collected data by using trapezoidal integration of the collected data and adds the calculated area of the extrapolated data using a series of computations.

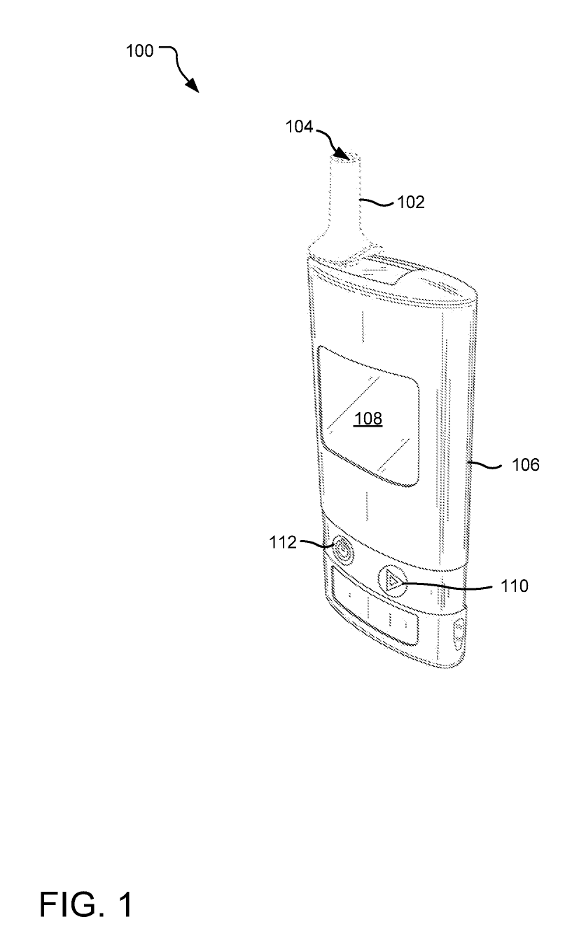

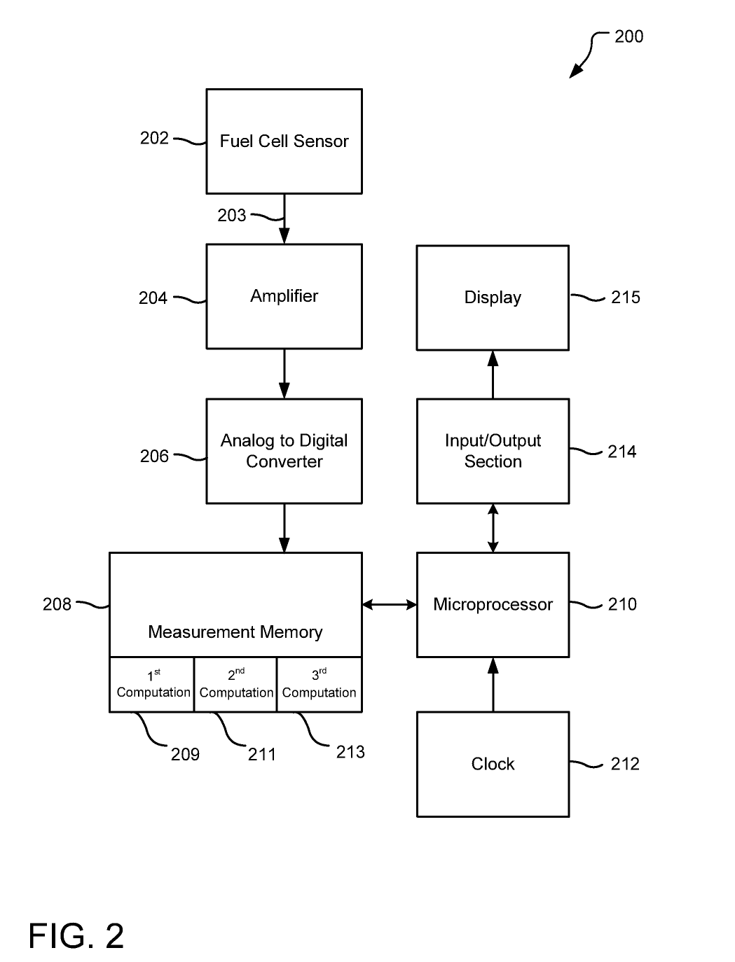

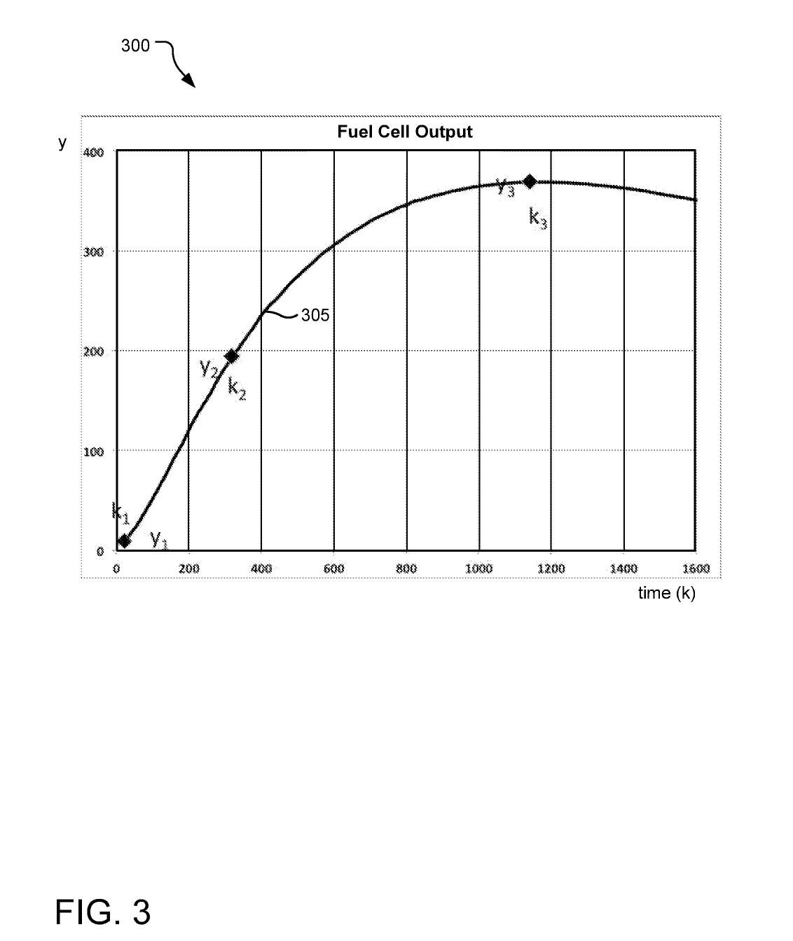

Referring to FIG. 1, a breath alcohol content device 100 is shown. During use of the device 100, a user breathes a fluid sample into the device 100 at a mouthpiece 102. The sample passes into the mouthpiece 102 through an inlet port 104 and may exit through the mouthpiece through an exhaust port (not shown) in a body 106 of the device 100, and an electrochemically convertible substance in the fluid sample is converted in an electrochemical sensor (e.g., fuel cell sensor 202 of FIG. 2) producing an electrical output. The electrical output is measured to produce sensor measurements, which are utilized in the disclosed methods for determining a quantity of the electrochemically convertible substance (described below and in FIG. 3) within the fluid sample. The determined quantity is displayed on a display 108 (e.g., in the form of a percentage of alcohol in the fluid sample or a breath alcohol content measurement). The device 100 may have user input buttons (e.g., input button 110), as well as an on/off power button 112. In another implementation, the display 108 may be a touch screen with input and/or power functions.

FIG. 2 is a block diagram that shows an example electronic circuit 200 of a fluid analysis apparatus (e.g., the breath alcohol content device 100 described in FIG. 1) utilized in operations for determining the quantity of an electrochemically convertible substance in a fluid sample (an example implementation is described in detail with regard to FIG. 3). A microprocessor 210 reads a clock 212 and records a start time upon introduction of a fluid sample into an electrochemical fuel cell sensor 202. An output 203 of the electrochemical fuel cell sensor 202 is amplified by an amplifier 204, and may be conditioned to reduce noise and scale amplitude. The conditioned analog output is converted into a digital signal for analysis by analog to digital converter 206.

At predefined periodic intervals beginning at the start time, the microprocessor 210 signals a measurement memory 208 to record sensor measurements. This signal causes the analog to digital converter 206 to latch the analog sensor output amplitude and the measurement memory 208 to store a digital representation of the output amplitude, which may be a current or voltage value. The microprocessor 210 reads sensor measurements from the measurement memory 208, and can perform operations (e.g., operations 300, including applying a first computation 209, a second computation 211, and/or a third computation 213 (stored in the measurement memory 208)) directed at determining quantity of the electrochemically convertible substance within the fluid sample, and write results of the operations back to the measurement memory 208 or to an external memory or storage device. An input/output (I/O) section 214 may be connected to one or more user-interface devices (e.g., a keyboard, a display unit 215, etc.), and communicate results from the measurement memory 208 or provide instructions to the electronic circuit 200.

A separate computing system (not shown) may also be used to implement some of the functional aspects of the electronic circuit 200 or add additional functional aspects. The computer system may be capable of executing a computer program product embodied in a tangible computer-readable storage medium to execute a computer process. Data and program files may be input to the computer system, which reads the files and executes the programs therein using one or more processors. Some of the elements of the computer system may include an I/O section, a central processing unit (e.g., processor), and a program memory. There may be one or more processors, such that the processor of the computer system comprises a single central processing unit, or a plurality of processing units, commonly referred to as a parallel processing environment. The computer system may be a conventional computer, a distributed computer, or any other type of computer. The described technology is optionally implemented in software loaded in memory stored on a storage unit, and/or communicated via a wired or wireless network link on a carrier signal, thereby transforming the computer system to a special purpose machine for implementing the described operations.

The I/O section in the computer system may be connected to one or more user-interface devices (e.g., a keyboard, a display unit, etc.) and/or storage units (e.g., storage drives or memory). Computer program products containing mechanisms to effectuate the systems and methods in accordance with the described technology may reside in the storage unit(s) of such a system.

A communication interface may be capable of connecting the computer system to an enterprise network via a network link, through which the computer system can receive instructions and data embodied in a carrier wave. When used in a local-area-networking (LAN) environment, the computer system is connected (by wired connection or wirelessly) to a local network through the network interface or adapter, which is one type of communications device. When used in a wide-area-networking (WAN) environment, the computer system typically includes a modem, a network adapter, or any other type of communications device for establishing communications over the wide area network. In a networked environment, program modules depicted relative to the computer system or portions thereof, may be stored in a remote memory storage device. It is appreciated that the network connections described are exemplary and other means of and communications devices for establishing a communications link between the computers may be used.

In an example implementation, a user interface software module and other modules may be embodied by instructions stored in memory (e.g., memory 208) and/or a storage unit and executed by a processor (e.g., microprocessor 210). Further, local computing systems, remote data sources and/or services, and other associated logic represent firmware, hardware, and/or software, which may be configured to assist in obtaining breath alcohol content measurements. A breath alcohol content computer process may be implemented using a general purpose computer and specialized software (such as a server executing service software), a special purpose computing system and specialized software (such as a mobile device or network appliance executing service software), or other computing configurations. In addition, breath alcohol content measurements and computations may be stored in a (e.g., memory 208) and/or a storage unit 912 and executed by a processor (e.g., microprocessor 210).

It should be understood that the breath alcohol content computer process may be implemented in software executing on a stand-alone computer system, whether connected to a breath alcohol content device or not. In yet another implementation, the breath alcohol content computer process may be integrated into a device (e.g., a breath alcohol content device).

The implementations of the invention described herein are implemented as logical steps in one or more computer systems. The logical operations of the present invention are implemented (1) as a sequence of processor-implemented steps executed in one or more computer systems and (2) as interconnected machine or circuit modules within one or more computer systems. The implementation is a matter of choice, dependent on the performance requirements of the computer system implementing the invention. Accordingly, the logical operations making up the embodiments of the invention described herein are referred to variously as operations, steps, objects, or modules. Furthermore, it should be understood that logical operations may be performed in any order, adding and omitting as desired, unless explicitly claimed otherwise or a specific order is inherently necessitated by the claim language.

Data storage and/or memory may be embodied by various types of storage, such as hard disk media, a storage array containing multiple storage devices, optical media, solid-state drive technology, ROM, RAM, and other technology. The operations may be implemented in firmware, software, hard-wired circuitry, gate array technology and other technologies, whether executed or assisted by a microprocessor, a microprocessor core, a microcontroller, special purpose circuitry, or other processing technologies. It should be understood that a write controller, a storage controller, data write circuitry, data read and recovery circuitry, a sorting module, and other functional modules of a data storage system may include or work in concert with a processor for processing processor-readable instructions for performing a system-implemented process.

For purposes of this description and meaning of the claims, the terms “computer readable storage media” and “memory” refer to a tangible data storage device, including non-volatile memories (such as flash memory, disc drives, and the like) and volatile memories (such as dynamic random access memory and the like). The computer instructions either permanently or temporarily reside in the memory, along with other information such as data, virtual mappings, operating systems, applications, and the like that are accessed by a computer processor to perform the desired functionality. The terms “computer readable storage media” and “memory” expressly do not include a transitory medium such as a carrier signal, but the computer instructions can be transferred to the memory wirelessly.

FIG. 3 is a graph 300 of example data values for fuel cell output of an example breath alcohol content device. When fuel cell output data curve 305 first exceeds a threshold y1, a starting time k1 is recorded and data collection proceeds. The threshold y1 is chosen above the system noise level to prevent false triggering for a zero sample (e.g., a zero alcohol sample).

From the start of data collection until a maximum fuel cell output value is reached, the output data values increase over time. A maximum value y3 is identified at the first time k3 when the curve 305 has reached the maximum value, and then drops in value. Eventually, subsequent values are all below the maximum value.

In some examples, a false positive or false maximum value may be present. For example, water vapor may be erroneously measured as a value for alcohol. For example, a sharp spike in the fuel cell output (not shown) may indicate a false measurement. In order to select accurate measurements for calculations, such false measurements are identified and discarded. In an example where repeated, identical maximum measurement values present, the first maximum value is used as the maximum value y3.

The average value of the first data sample y1 and the peak data sample y3 is calculated with the following equation: y2=(y1+y3)±2. The data samples are then searched to find the time when the data first exceeds the value y2. The time k2 and the data value y2 are recorded. The three data sample pairs (k1, y1), (k2, y2), (k3, y3) from the measurement data are then used in calculations with a first computation.

The first computation may include the following operations: 1) select the time to start collecting measurement data; 2) detect the time of the occurrence of the maximum; 3) collect data from the starting time to the time of the maximum; and 4) calculate the parameters needed for a second computation using a first curve fit equation, while detecting any error conditions. The first computation is robust against shifts in the starting value and starting time, shifts in the average value and average time, as well as errors in the peak value and peak time.

Collected data points between y1 and y3 are used to calculate the parameters (x0, x1 and x2) for an exponential curve fit to a version of the fuel cell response equation y=x0 (e^{x1 k}−e^{x 2 k}) where y is the output signal amplitude and k represents time (referred to herein as the “first curve fit equation”). In this equation, “y” represents the quantity of the electrochemically convertible substance in the fluid sample, “x0” represents the amplitude factor, “x1” represents the fuel cell discharge factor and “x2” represents the initial reaction factor. In various implementations, x0 is positive and x1 and x2 are negative integer values. The associated time constants are −1/x1 and −1/x2.

In various implementations, the −1/x1 time constant is substantially larger than the −1/x2 time constant. This causes the first exponential term to decay at a slower rate than the second exponential term. The first term starts at a value of 1 and drops in amplitude but not significantly for the purposes of the first computation.

A simplification may be made by setting the first exponential term in the first curve fit equation to 1. In some implementations, this simplification causes an acceptable inaccuracy in the curve fit as the error has negligible effect on a final area under the curve 305 computation. The remaining parameters are sufficiently accurate for their intended purposes. The simplified equation is y=x0(1−e^{x2k}). The three data sample pairs are used to determine the values for x0 and x2. The data sample pairs and the calculated x0 and x2 parameters are used to identify fault conditions.

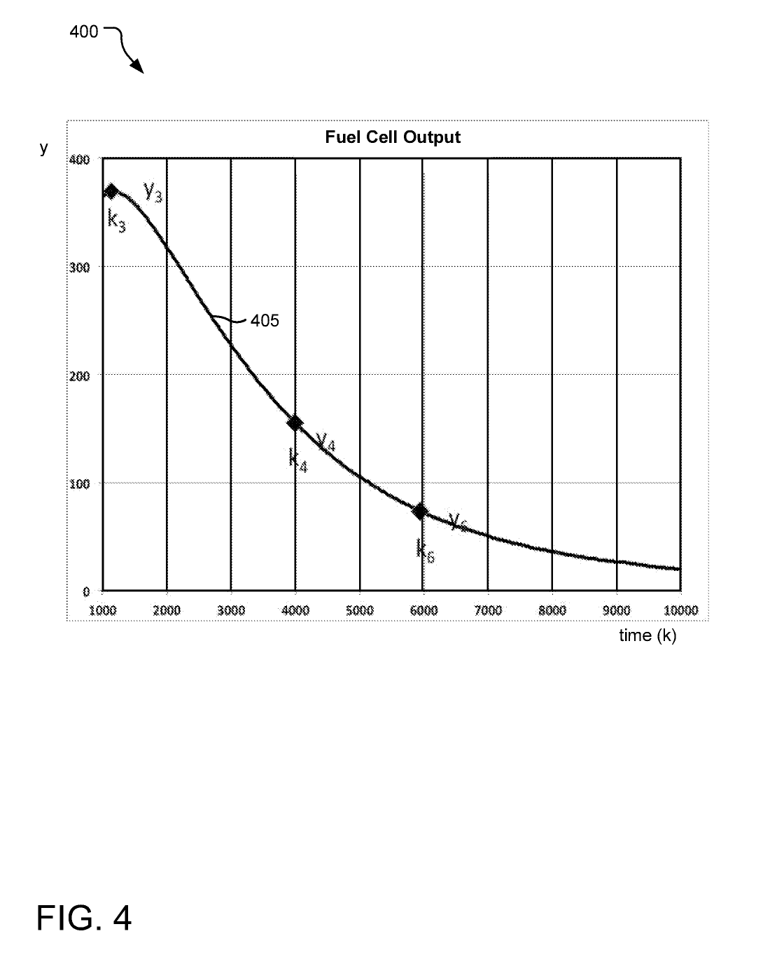

FIG. 4 is a second graph 400 of example data values for fuel cell output of an example breath alcohol content device. In one implementation, the graph 400 contains a data pair (k3, y3) that matches data pair (k3, y3) of graph 300 of FIG. 3 and data pairs (k4, y4) and (k5, y5) subsequent in time to the data illustrated in FIG. 3.

A second computation, which may involve several steps and equations described in detail below, determines when to truncate data collection and to curve fit a function to the truncated data following the peak (k3, y3) of data curve 400 using a second curve fit equation. The second computation may then calculate the remaining area under the extrapolated tail of the data values. The choice of the truncation time affects the total measurement time and the accuracy of the result.

As shown in FIG. 4, a graph 400 of data versus time values for fuel cell output illustrates the peak value y3, which was identified using the first computation. In one implementation, the second computation uses a truncation threshold based on the peak value y3. For example, a truncation threshold may be set at 20% of the peak value. While data continues to be collected, the data is compared to the 20% truncation threshold and when data falls below the truncation threshold, data collection is stopped (see e.g., at data point (k6, y6)).

The determination of when to truncate may be based on a preselected time (vertical line) or preselected output value (horizontal line) or using another more complicated computation. With the resulting truncated data, the second computation may calculate an estimated area under the extrapolated curve 400 out to infinity. The accuracy of the extrapolation depends on a number of factors and will have some residual error. The residual error can be compensated for using a calibration factor. If the ratio of the error in the extrapolated tail to the total area can be kept constant independent of amplitude scaling and time scaling, then the calibration factor will be more independent of scaling as well. This independence of scaling leads to a reduction in compensation factors for factors affecting the shape of the curve due to temperature variations, sensor variations, sensor aging, sampling cycle variations, concentration, and other factors.

The data curve 400 in FIG. 4 is approximately exponential in shape with the following equation: y=k(e^{-at}−e^{-bt}). A first threshold is chosen to allow time for the fast exponential to decay to a low value and to allow the curve fitting constants to stabilize to values more representative of the tail dynamics. A threshold is chosen at approximately 45% of the peak value y3. The value y4 close to the 45% threshold is found at corresponding time k4. The remaining data curve from time k4 is calculated with the following equation: y=k(e^{-at}−0)=ke^{-at}. A second threshold used to truncate the data collection may be set at 20%.

For a simple exponential decay, it will be shown that the ratio of the area of the extrapolated tail from time k6 on versus the total area from time k4 on is dependent on only the threshold ratio of 20%±45% and therefore is independent of time or amplitude scaling. The total area of the curve from k4 to infinity may be calculated with the following equation:

$$\begin{matrix}y = {\int }_{k 4}^{∞} k {e}^{- a t} \\ = {\vert \frac{k {e}^{- a t}}{- a} \vert}_{k 4}^{∞} \\ = \frac{k {e}^{- a ∞}}{- a} - \frac{k {e}^{- a k 4}}{- a} \\ = 0 - \frac{k {e}^{- a k 4}}{- a} \\ = \frac{k {e}^{- a k 4}}{a}\end{matrix}$$

Similarly, the area of the extrapolated tail is calculated with the following equation:

$$\frac{k {e}^{- a k 6}}{a}$$

The following equation represents taking the ratio of the tail area from k6 on to the total area from k4 on:

$$\frac{k {e}^{- a k 6}}{a} / \frac{k {e}^{- a k 4}}{a}$$

Cancelling “a” from both denominators yields the following equation:

ke^{-ak6}/ke^{-ak4}=y⁶/y4

The analysis shows the ratio of y6/y4 equals the ratio of the areas and is independent of scaling in amplitude or time for a perfect exponential decay. The data curve 400 illustrated in FIG. 4 is close to but not a perfect exponential decay. An exponential curve fit results in significant error between the actual tail area and the extrapolated tail area. In order to improve the fit, an exponential function is chosen with higher order terms and an improved curve fit is performed.

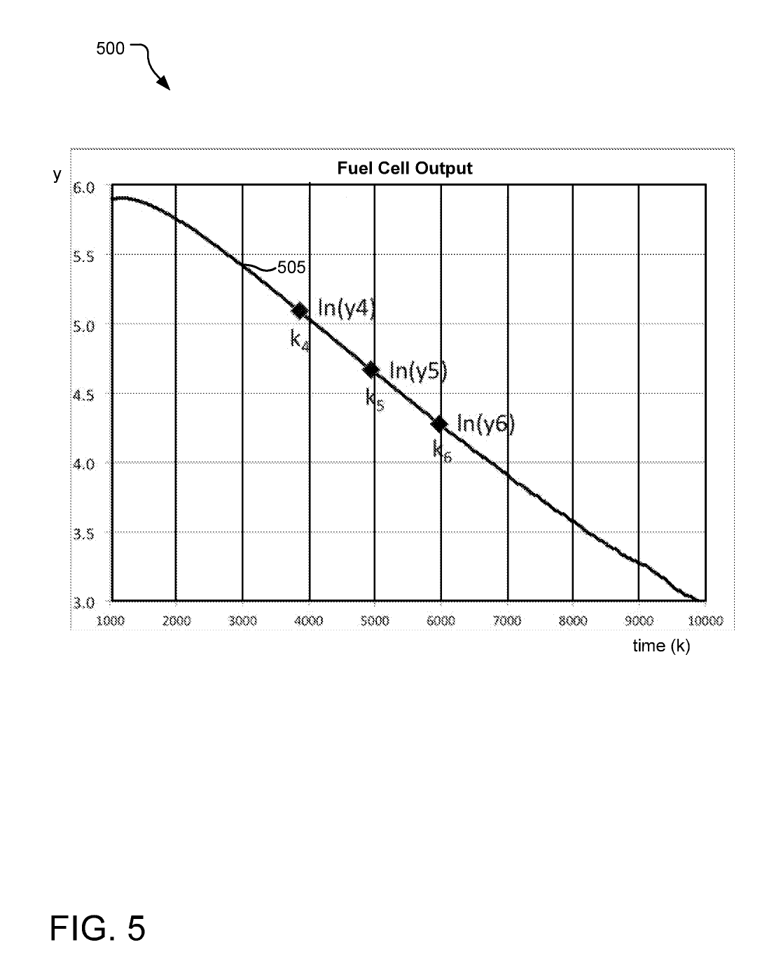

FIG. 5 is a third natural logarithmic graph 500 of example data values for fuel cell output of an example breath alcohol content device. In one implementation, the graph 500 illustrates the natural logarithm for the data illustrated in graph 400 of FIG. 4. To improve computation fitting robustness, the curve fitting discussed above with regard to FIG. 4 may be performed in the natural logarithm domain instead of a direct curve fit to the data.

First, the natural logarithm for each data point (k4, ln(y4)) and (k6, ln(y6)) is calculated, as shown in graph 500 of data versus time values for the fuel cell output. Next, a polynomial fit is calculated. Initial estimates for the quadratic terms of the polynomial are calculated by selecting three data points and solving for the quadratic polynomial fit. The three data points include the points at y4 and y6 and a data point located half way between y4 and y6 (i.e., (k5, ln(y5)).

After solving for the initial estimate of the polynomial quadratic coefficients, the second computation then performs adaptive steps to refine all of the coefficients. The adaptive steps are performed using a Gauss-Newton computation applied as part of the second computation to a least mean square error polynomial fit to the natural logarithm of the data between k4 and k6. The Gauss-Newton computation is repeated until the coefficients settle to final values. The choice of performing a least mean square polynomial fit in the logarithm of data domain using a Gauss-Newton computation results in a computation that is very robust and often converges in one or two steps.

In another implementation, it is possible to perform a least mean square fit of an exponential with polynomial terms directly in the data domain using a simple Gauss-Newton computation method, but the computation is only locally convergent. This implementation may use more advanced added computation methods to search to the local convergence region and/or a coarse computation that calculates a starting point inside the local convergence region. Operating in the logarithm of data domain has a wider convergence region and faster convergence and uses less overall computing power.

After performing the adaptive steps to refine the equation coefficients, the next step in the second computation is computing the area of the tail of curve 400 of FIG. 4. The area is calculated by taking the exponential of the fitted polynomial function evaluated at each time increment after k6 and performing trapezoidal area summation in one of three ways. If the second order coefficient is positive then the area summation may be continued until the polynomial reaches a minimum, and is then terminated. In another example, where the second order coefficient x2 has a value of 0 or is negative, the area summation is terminated when the polynomial crosses 0. There may be a small residual error left since the function is not a perfect fit to the data.

A third computation is used and run in parallel with the first computation and the second computation while data collection is occurring. The third computation accumulates the trapezoidal area as each data point is recorded until, for example, a time k6 (or cut-off time) shown in FIGS. 4 and 5. The third computation then computes the total area by adding the accumulated area to the estimated area of the tail from the second computation.

Various equation solving software programs may be used with one or more computations in the disclosed technology. For example, the Microsoft® Office Excel® software comprises an add-in program entitled, “Solver,” which may be utilized for such calculations. The data of the sensor measurements may be input into the Solver program, or a custom equation solving program software, to curve fit the equation.

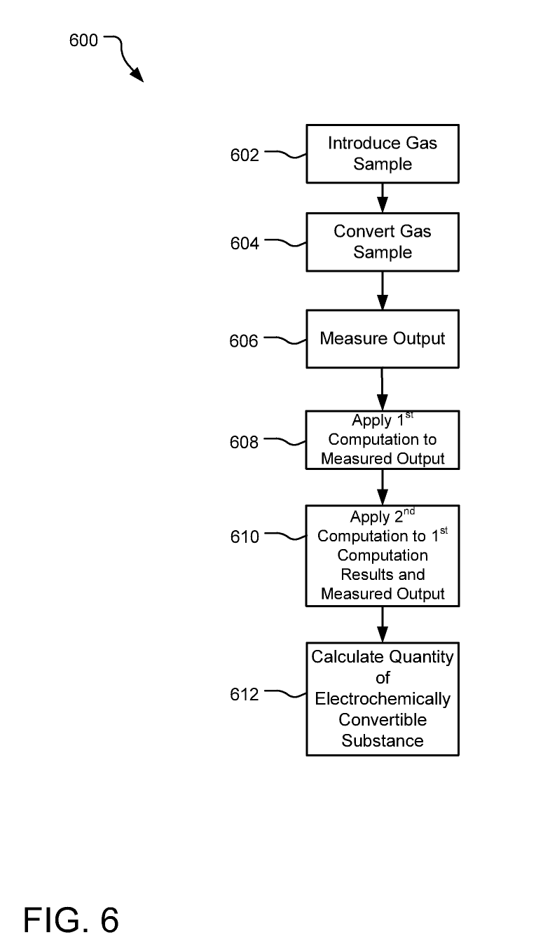

FIG. 6 is flowchart of example operations 600 for determining the quantity of an electrochemically convertible substance in a fluid sample. Introducing operation 602 introduces a fluid sample into an electrochemical sensor. Introducing operation 602 may be performed by a user breathing into a breath alcohol sensing device, for example. The user's breath passes adjacent to the electrochemical sensor within the breath alcohol sensing device. Conversion operation 604 electrochemically converts at least a portion of the fluid sample into an electrical output from the electrochemical sensor. In various implementations, the electrochemical sensor is a fuel cell device that uses alcohol content in the fluid sample to vary the output of the fuel cell. Measuring operation 606 measures the electrical output from the electrochemical sensor on a periodic basis to produce sensor measurements. The periodic sensor measurements are stored in a memory for further analysis via the first, second, and third computations discussed in detail herein.

A first applying operation 608 applies a first computation to the measured and stored sensor output. The first computation may, for example, select a time to start collecting measurement data from the electrochemical sensor, detect a time of occurrence of a sensor output maximum, collect data from the selected start time to the detected maximum time, and calculate parameters for a second computation using a first curve fit equation (see FIG. 2), all while detecting and compensating for any error conditions that may be present.

A second applying operation 610 applies a second computation to an output of the first computation and the measured and stored sensor output. The second computation may, for example, determine when to truncate data collection, apply second curve fit equation to the truncated data following the peak of the data curve, and calculate the remaining area under the extrapolated tail of the data values.

A calculating operation 612 sums the results of the first computation and the second computation. As a result, the calculating operation may estimate the total area under the electrochemical sensor output curve with only a portion of the actual data available and in a fraction of the time needed to measure substantially all of the electrochemical sensor output data.

A quantity of the electrochemically convertible substance within the fluid sample is calculated using the summed area under the electrochemical sensor output curve. Applying the first, second, and third computations in the manner disclosed herein may utilize a small fraction (e.g., less than 10%) of the processing power and software functionality typically used in a full curve fitting software package. As a result, the methods disclosed herein may be implemented on a relatively small and inexpensive package (e.g., within a breath alcohol detection device).

The above specification, examples, and data provide a complete description of the structure and use of example implementations of the invention. Since many implementations of the invention can be made without departing from the spirit and scope of the invention, the invention resides in the claims hereinafter appended. Furthermore, structural features of the different implementations may be combined in yet another implementation without departing from the recited claims. The implementations described above and other implementations are within the scope of the following claims.

What is claimed is:

1. A method of determining a quantity of an electrochemically convertible substance in a fluid sample, the method comprising:
introducing the fluid sample into an electrochemical sensor, wherein at least a portion of the fluid sample is electrochemically converted to produce an electrical output from the electrochemical sensor;
measuring the electrical output from the electrochemical sensor on a periodic basis to produce sensor measurements;
inputting a first subset of the sensor measurements into a first computation to yield first computation analysis results;
inputting a second subset of the sensor measurements and the first computation analysis results into a second computation to yield second computation analysis results; and
calculating the quantity of the electrochemically convertible substance in the fluid sample by applying a third computation to the first computation analysis results and the second computation analysis results.

2. The method of claim 1, further comprising solving for a plurality of shaping constants in the second computation.

3. The method of claim 1, further comprising applying a Gauss-Newton computation to yield the second computation analysis results.

4. The method of claim 1, wherein the measuring the electrical output of the electrical sensor is performed at predetermined time intervals.

5. The method of claim 1, wherein the inputting the first subset of the sensor measurements operation and the inputting the second subset of the sensor measurements operation each includes:
solving an arithmetic equation y=x0 (e^{x1 k}−e^{x2 k}), wherein y represents the quantity of the electrochemically convertible substance in the fluid sample, “x0” represents the amplitude factor, “x1” represents the fuel cell discharge factor, and x2″ represents the initial reaction factor.

6. The method of claim 4, wherein an electrochemical sensor output curve is approximated by matching the arithmetic equation to the sensor measurements.

7. The method of claim 5, wherein an area under the electrochemical sensor output curve is calculated by applying the third computation to the first computation analysis results and the second computation analysis results.

8. The method of claim 4, wherein “x1” fuel cell discharge factor is approximated using a value of zero in the inputting the first subset of the sensor measurements operation.

9. The method of claim 1, wherein the electrochemically convertible substance is an alcohol.

10. The method of claim 1, wherein the inputting the second subset of the sensor measurements operation is performed using an equation solving software program.

11. The method of claim 1, wherein the calculating operation further comprises:
comparing a calculated initial reaction factor to a predetermined reaction factor associated with a predetermined reactant; and
comparing a discharge factor to the predetermined discharge factor associated with the predetermined reactant.

12. A device, comprising:
an electrochemical sensor configured to convert an electrochemically convertible substance in a fluid sample to an electrical output on contact with the electrochemical sensor;
a memory configured to store the series of electrochemical sensor measurements, a first curve fit computation, and a second curve fit computation; and
a microprocessor configured to measure the electrical output of the electrochemical sensor to produce sensor measurements, apply a first computation to a first subset of the sensor measurements to yield first computation analysis results, apply a second computation to a second subset of the sensor measurements and to the first computation analysis results, and calculate the quantity of the electrochemically convertible substance in the fluid sample by applying a third computation to the first computation analysis results and second computation analysis results.

13. The device of claim 12, further comprising:
a clock configured to serve as a reference point for the series of electrochemical sensor measurements;
a mouthpiece configured to allow the user to breath the fluid sample into the device;
an inlet port configured to allow the fluid sample to enter the device from the mouthpiece;
an exhaust port configured to allow the fluid sample to exit the mouthpiece.

14. The device of claim 12, wherein the device is a handheld breath alcohol testing device.

15. The device of claim 12, wherein the electrochemically convertible substance is an alcohol.

16. The device of claim 12, further comprising a display configured to display the calculated quantity of the electrochemically convertible substance within the fluid sample.

17. The device of claim 12, wherein the electrochemically convertible substance is ethanol.

18. The device of claim 12, further comprising:
an amplifier configured to amplify the output of the electrochemical fuel cell sensor; and
an analog to digital converter configured to digitize the amplified analog output from the electrochemical fuel cell sensor prior to storage in memory.

19. One or more computer readable storage media storing computer-executable instructions in memory and executable to perform a computer process, the computer process comprising:
introducing a fluid sample into an electrochemical sensor, wherein at least a portion of the fluid sample is electrochemically converted to produce an electrical output from the electrochemical sensor;
measuring the electrical output from the electrochemical sensor on a periodic basis to produce sensor measurements;
inputting a first subset of the sensor measurements into a first computation to yield first computation analysis results;
inputting a second subset of the sensor measurements and the first computation analysis results into a second computation to yield second computation analysis results; and
calculating the quantity of the electrochemically convertible substance in the fluid sample by applying a third computation to the first computation analysis results and the second computation analysis results.

20. The one or more computer readable storage media of claim 19, wherein operation of calculating the quantity of the electrochemically convertible substance comprises:
calculating an arithmetic equation y=x0 (e^{x1 k}−e^{x2 k}), wherein y represents the quantity of the electrochemically convertible substance in the fluid sample, “x0” represents the amplitude factor, “x1” represents the fuel cell discharge factor, and x2″ represents the initial reaction factor.
